(19) United States
(12) Patent Application Publication
(10) Pub. No.: US 2021/0060136 A1
(43) Pub. Date: Mar. 4, 2021

(54) USE OF HEMOGLOBIN FROM ANNELIDS AS BACTERICIDE, IN PARTICULAR FOR PREVENTING AND/OR TREATING A PERIODONTAL DISEASE

(71) Applicant: HEMARINA, Morlaix (FR)

(72) Inventor: Franck ZAL, Ploujean Morlaix (FR)

(21) Appl. No.: 16/962,906

(22) PCT Filed: Jan. 16, 2019
(86) PCT No.: PCT/EP2019/051076

§ 371 Date: Jul. 17, 2020

(30) Foreign Application Priority Data
Jan. 17, 2018 (FR) 1850365

(51) Int. Cl. A61K 38/42; A61K 9/06; A61K 8/64; A61P 1/02; A61Q 11/00; A61K 38/17

(52) U.S. Cl. CPC A61K 38/42; A61K 9/06; A61K 38/1767; A61P 1/02; A61Q 11/00; A61K 8/64

(57) ABSTRACT

The present invention relates to the use of a molecule chosen from among the oxygen transporters of marine invertebrate animals, preferably from a globin, a globin protomer, or an extracellular hemoglobin of Annelids, as a bactericide, in particular for preventing and/or treating periodontal disease.

The present invention relates to the use of a molecule chosen from among oxygen transporters of marine invertebrate animals, preferably chosen from among a globin, a globin protomer, or an extracellular hemoglobin of Annelids, as a bactericidal agent. The present invention also relates to the use of a molecule chosen from among oxygen transporters of marine invertebrate animals, preferably chosen from among a globin, a globin protomer, or an extracellular hemoglobin of Annelids, for preventing and/or treating periodontal disease.

Periodontal diseases are diseases of infectious origin (bacteria), which affect and destroy the supporting tissues of the teeth forming the periodontium. The periodontium is made up of four tissues: the gum, the alveolar bone, the alveolo-dental ligament, and the cementum. When periodontal disease is limited to the gum, it is called gingivitis. When it affects the entire periodontium, it is called periodontitis.

Periodontal diseases develop quite slowly over several decades. They are mainly due to dental plaque (i.e. accumulation of food debris and bacteria) which adheres to the surface of the tooth located under the edge of the gum. Tartar is the calcification of this dental plaque. It is colonized by pathogenic bacteria. Stagnation of bacteria in dental plaque is the cause of an inflammatory reaction on the gums and bone, causing their destruction over the course of months and years.

As mentioned above, the formation of a biofilm is an essential step in the onset of periodontal disease. It is done in a hierarchical manner, with first the colonization of the oral surfaces by primary colonizers thanks to ligands and nutrients present in the environment, then by secondary or late colonizers like Porphymonas gingivalis (P. gingivalis) and Treponema denticola (T. denticola) whose installation in the biofilm is favored by the primary colonizers. Certain periodontopathogenic species have been identified as being mainly associated with periodontal diseases, and form what is called the “red complex”: these are P. gingivalis, T. denticola and T. forsythia.

Among the primary colonizers is Streptococcus gordonii. This bacteria is one of the commensal bacteria in the oral environment. It is a Gram-positive shell belonging to the phylum Firmicutes, optionally aerobic-anaerobic.

P. gingivalis belongs to the phylum Bacteroidetes; it is a Gram-negative, strictly anaerobic, aerotolerant, proteolytic, and encapsulated coccobacillus. P. gingivalis is mainly found in the oral environment at the level of the subgingival sites, but may be isolated in small numbers in saliva and on the oral mucosa.

T. denticola, like P. gingivalis, is a bacterium belonging to the “red complex” and is therefore responsible for periodontitis. It belongs to the phylum Spirochaetaceae; it is a Gram negative spirochete, strictly anaerobic, motile.

Metabolic cooperation between P. gingivalis and T. denticola has also been demonstrated (Meuric et al., 2013; Yamada et al., 2005) involving proteases which allow the release by one species of substrates that are usable by the other species.

The treatments for periodontal diseases are distinct and more or less complex depending on the stage of the disease: in the case of gingivitis, scaling and good dental hygiene, possibly supplemented by antibiotic therapy, are generally sufficient. When periodontal disease progresses to periodontitis, however, the buildup of plaque between the gum and the tooth results in loss of gum attachment and resorption of the bone that surrounds the tooth. This phenomenon is responsible for the formation of periodontal pockets between the gum, the tooth and the bone. There is a beginning of destruction of the underlying bone. In addition, the formation of these pockets promotes the accumulation of dental plaque which worsens the resorption of the bone. We then enter a vicious circle. In this case, the treatment consists of performing a root planning (if necessary under local anesthesia) which aims to remove dental plaque and tartar located under the gum. The objective of this treatment is to cause a re-attachment between the gum and the surfaces of the roots previously exposed. Periodontal surgeries may also be considered.

Gingivitis may regress completely. Periodontitis may be stabilized. But only strict dental hygiene will prevent recurrence.

There is therefore a need for effective treatment of bacterial diseases, and, in particular, periodontal, and, especially, periodontitis.

The inventors have now discovered that, surprisingly, the extracellular hemoglobin of Annelids, especially Arenicola marina, has anti-biofilm activity. In fact, as demonstrated in the example, it appears that the extracellular hemoglobin of Arenicola marina has a bactericidal effect on planktonic cultures of P. gingivalis and T. denticola. In addition, S. gordonii is capable of more significantly inhibiting the growth of P. gingivalis in the presence of this hemoglobin. Finally, in an in vitro model of oral biofilm composed of S. gordonii (primary colonizer), P. gingivalis and T. denticola (periodontopathogens), this hemoglobin seems to promote the detachment of the cells of P. gingivalis and T. denticola. In conclusion, the extracellular hemoglobin of Annelids, in particular of Arenicola marina, seems to exhibit bactericidal activity with respect to Gram-negative bacteria that are pathogenic, in particular for humans.

In addition, the extracellular hemoglobin of Annelids, in particular Arenicola marina, exhibits bactericidal activity against strictly anaerobic bacteria, in particular by the supply of oxygen.

The present invention thus relates to the use of a molecule chosen from among the oxygen transporters of marine invertebrate animals, preferably chosen from among a globin, a globin protomer, or an extracellular hemoglobin of Annelids, as a bactericidal agent with respect to Gram negative bacteria that are pathogenic for humans. Pathogenic bacteria are bacteria responsible for diseases even in healthy people.

Gram negative bacteria are identified by the Gram staining technique: thick-walled bacteria are colored purple and called “Gram positive”, while thin-walled bacteria are colored red and called “Gram negative”.

The molecule chosen from among the oxygen transporters of marine invertebrate animals, and preferably chosen from among a globin, a globin protomer, or an extracellular hemoglobin of Annelids, according to the invention, may thus be used as an antibiotic against Gram-negative bacteria that are pathogenic for humans.

Gram-negative bacteria are chosen, in particular, from among P. gingivalis, T. denticola, T. forsythia, G. meningitidis, E. coli, H. influenzae, P. aeruginosa, B. pertussis, L. pneumoniae and H. pylori.

Preferably, Gram-negative bacteria that are pathogenic to humans are strictly anaerobic. More preferably, it is P. gingivalis and/or T. denticola. Such bacteria are present, in particular, in the periodontal pockets. These pockets are hypoxic, and therefore provide conditions favorable to the proliferation of these strictly anaerobic bacteria.

The molecule chosen from among the oxygen transporters of marine invertebrate animals, and preferably chosen from among a globin, a globin protomer, or an extracellular hemoglobin of Annelids, according to the invention, may also be used to treat various disorders induced by such bacteria.

In particular, it may be used to prevent and/or treat bad breath (or halitosis), and/or to whiten teeth.

The present invention also relates to the use of a molecule chosen from among the oxygen transporters of marine invertebrate animals, preferably chosen from among a globin, a globin protomer, or an extracellular hemoglobin of Annelids, for preventing and/or treating periodontal disease.

The present invention thus also relates to the use of a molecule chosen from among oxygen transporters of marine invertebrate animals, and preferably chosen from among a globin, a globin protomer, or an extracellular hemoglobin of Annelids, for preventing and/or treating halitosis.

The present invention also relates to the use of a molecule chosen from among oxygen transporters of marine invertebrate animals, preferably chosen from among a globin, a globin protomer, or an extracellular hemoglobin of Annelids, for whitening teeth.

The present invention also relates to the use of a molecule chosen from among the oxygen transporters of marine invertebrate animals, and preferably chosen from among a globin, a globin protomer, or an extracellular hemoglobin of Annelids, for treating wounds caused by Gram-negative bacteria that are pathogenic to humans.

Preferably, periodontal disease is chosen from among gingivitis, periodontitis, periodontal recessions, and periodontal abscesses.

The molecule according to the invention is chosen from among oxygen carriers of marine invertebrate animals.

Preferably, it is chosen from among a globin of Annelids, a globin protomer of Annelids, or an extracellular hemoglobin of Annelids.

“Oxygen transporter” means any molecule capable of reversibly transporting oxygen from the environment to target cells, tissues or organs.

The oxygen transporter according to the invention comes from marine invertebrate animals.

Among marine invertebrate animals, may be mentioned, in particular, Annelids, molluscs, brachiopods and crustaceans.

Preferably, the oxygen transporter of marine invertebrate animals is a metalloprotein.

More preferably, the oxygen transporter of marine invertebrate animals is chosen from among hemoglobins, globin protomers, globins, hemerythrins, myohemerythrins, chlorocruorins, erythrocruorins and hemocyanins.

Hemoglobins, globin protomers and globins preferably come from Annelids.

Hemerythrines and myohemerythrins preferably come from brachiopods or Annelids (Polychetes).

The chlorocruorins and erythrocruorins preferably come from Annelids Polychetes.

Finally, the hemocyanins preferably come from molluscs or crustaceans.

The extracellular hemoglobin of Annelids is present in the three classes of Annelids: Polychetes, Oligochaetes and Achetes. We talk about extracellular hemoglobin because it is naturally not contained in a cell, and may, therefore, circulate freely in the blood system without the need for chemical modification to stabilize or make it functional.

The extracellular hemoglobin of Annelids is a giant biopolymer with a molecular weight between 2000 and 4000 kDa, consisting of approximately 200 polypeptide chains between 4 and 12 different types, which are generally grouped into two categories.

The first category, comprising 144 to 192 elements, groups together the so-called “functional” polypeptide chains which carry an active heme-type site and are capable of reversibly binding oxygen; these are globin-type chains (eight types in total for Arenicola marina hemoglobin: a1, a2, b1, b2, b3, c, d1 and d2), whose masses are between 15 and 18 kDa. They are very similar to the a and p type chains of vertebrates.

The second category, comprising 36 to 42 elements, groups together the so-called “structure” or “linker” polypeptide chains having little or no active site but allowing the assembly of subunits called twelfths or protomers. There are two types of linkers, L1 and L2.

Each hemoglobin molecule consists of two superimposed hexagons called hexagonal bilayer, wherein each hexagon is itself formed by the assembly of six subunits (dodecamer or protomer) in the form of a drop of ‘water. The native molecule is made up of twelve of these subunits (dodecamer or protomer). Each subunit has a molecular mass of about 250 kDa, and constitutes the functional unit of the native molecule.

Preferably, the extracellular hemoglobin of Annelids is chosen from among the extracellular hemoglobins of Annelid Polychetes and the extracellular hemoglobins of Annelid Oligochaetes. Preferably, the extracellular hemoglobin of Annelids is chosen from extracellular hemoglobins of the Lumbricidae family, extracellular hemoglobins of the Arenicolidae family, and extracellular hemoglobins of the Nereididae family. Even more preferably, the extracellular hemoglobin of Annelids is chosen from the extracellular hemoglobin of Lumbricus terrestris, the extracellular hemoglobin of Arenicola sp and the extracellular hemoglobin of Nereis sp, more preferably the extracellular hemoglobin of Arenicola marina or of Nereis virens. The arenicola Arenicola marina is a polychaete annelid worm living mainly in sand.

According to the invention, the globin protomer of the extracellular hemoglobin of Annelids constitutes the functional unit of the native hemoglobin, as indicated above. Finally, the globin chain of the extracellular hemoglobin of Annelids may be chosen, in particular, from globin chains of the Ax and/or Bx type of extracellular hemoglobin of Annelids.

The extracellular hemoglobin of Annelids, its globin protomers and/or its globins do not require a cofactor to function, unlike the mammalian hemoglobin, in particular human. Finally, the extracellular hemoglobin of Annelids, its globin protomers and/or its globins having no blood typing, make it possible to avoid any problem of immunological reaction. The extracellular hemoglobin of Annelids, its globin protomers and/or its globins exhibit intrinsic superoxide dismutase (SOD) activity. Consequently, this intrinsic antioxidant activity does not require any antioxidant to function, unlike the use of a mammalian hemoglobin for which the antioxidant molecules are contained inside the red blood cell and are not linked to hemoglobin. This SOD activity makes the molecule particularly effective in the treatment of halitosis.

The extracellular hemoglobin of Annelids, its globin protomers and/or its globins may be native or recombinant.

According to the invention, the oxygen transporter of marine invertebrate animals, preferably globin, the globin protomer or the extracellular hemoglobin of Annelids, is preferably present in a composition comprising a buffer solution. According to the invention and as indicated in the examples, the oxygen transporter of marine invertebrate animals, preferably globin, the globin protomer, or the extracellular hemoglobin of Annelids, is preferably present in a composition devoid of hydrocolloid, preferably in a liquid composition devoid of hydrocolloid (buffer solution devoid of hydrocolloid). Preferably, such a composition consists solely of the oxygen transporter of marine invertebrate animals, preferably a globin, a globin protomer or an extracellular hemoglobin of Annelids, and a buffer solution.

The formulation of the oxygen transporter of marine invertebrate animals, preferably globin, the protomer of globin, or the extracellular hemoglobin of Annelids, in liquid form has the advantage of being more easily administered.

Said buffer solution creates an adequate salt environment for the transporter and, in particular, hemoglobin, its protomers and its globins, and thus allows the maintenance of the quaternary structure, and, therefore, of the functionality of this molecule. Thanks to the buffer solution, the transporter and, in particular, the hemoglobin, its protomers, and its globins are capable of ensuring their oxygenation function.

The buffer solution according to the invention is preferably an aqueous solution comprising salts, preferably chloride, sodium, calcium, magnesium and potassium ions, and gives the composition according to the invention a pH of between 6.5 and 7.6; its formulation is similar to that of a physiologically injectable liquid. Under these conditions, the extracellular hemoglobin of Annelids, its globin protomers, and its globins, remain functional.

In the present description, the pH is understood to be at ambient temperature (25° C.), unless otherwise stated.

Preferably, the buffer solution is an aqueous solution comprising sodium chloride, calcium chloride, magnesium chloride, potassium chloride, as well as sodium gluconate and sodium acetate, and has a pH of between 6.5 and 7.6, preferably equal to 7.1±0.5, preferably approximately 7.35. More preferably, the buffer solution is an aqueous solution comprising 90 mM NaCl, 23 Mm Na-gluconate, 2.5 mM CaCl₂, 27 mM Na-acetate, 1.5 mM MgCl₂, 5 mM KCl, and has a pH of 7.1±0.5, which may contain between 0 and 100 mM of antioxidant such as ascorbic acid and/or reduced glutathione.

Preferably, the composition is administered to the subject parenterally, preferably by injection or infusion; or, topically, on the gums and/or teeth.

The composition may be administered to the subject in the form of a gel. This gel may be present, for example, in a mouthpiece which is applied to all of the teeth of the upper jaw or the lower jaw. It may also be applied directly on teeth and/or gums. Such a gel thus comprises at least one oxygen transporter of marine invertebrate animals, preferably a globin, a globin protomer, or an extracellular hemoglobin of Annelids, and a hydrophilic matrix. The hydrophilic matrix comprises, in particular, at least one gelling agent. Such a gel preferably comprises at least one oxygen transporter of marine invertebrate animals, preferably a globin, a globin protomer, or an extracellular hemoglobin of Annelids, a buffer solution, and a gelling agent which is not a hydrocolloid. Preferably, the gelling agent is chosen from among xanthan gum, guar gum and its derivatives (hydroxypropylguar, for example).

Finally, it may be applied using a syringe, for example in the case of periodontal pockets.

Preferably, the composition comprising the oxygen transporter of marine invertebrate animals, preferably hemoglobin, its protomers or its globins, and the buffer solution is administered as such. In fact, in this case, hemoglobin, its protomers or its globins, is present in a composition comprising a buffer solution, preferably an aqueous solution comprising salts, and conferring a pH of between 6.5 and 7.6 on the composition. Preferably, the composition contains only the oxygen transporter of marine invertebrate animals, preferably hemoglobin, its protomers, or its globins, and a buffer solution consisting of an aqueous solution comprising salts, and conferring a pH between 6.5 and 7.6 on the composition. The administration dosage is therefore quite simple and effective.

The invention is described in more detail in the following examples. These examples are provided for illustration purposes only, and are not limitative.

### EXAMPLES

Materials and Methods

1) Bacterial Strains and Culture Conditions

The strains used in this study are the strain of Porphyromonas gingivalis TDC60 (JCM19600) from the RIKEN BioResource Center, Japan, the strain of Treponema denticola ATCC® 35405 and the strain Streptococcus gordonii Challis CH1 ATCC®35105 (Lunsford and London, 1996). The strains of T. denticola and P. gingivalis were cultivated in liquid medium defined for their growth, MMBC-S medium, the composition of which is presented in Table 1.

TABLE 1
Composition of the medium defined for the growth of
Porphyromonas gingivalis and Treponema denticola (MMBC-S)

| | Final concentration | | Final concentration | |
|---|---|---|---|---|
| Elements | (mg/ml) | | (mM) | |
| NaH2PO4 | 1380 | mg/l | 10 | mM |
| KCl | 745 | mg/l | 10 | mM |
| MgCl2, 7H2O | 2033 | mg/l | 10 | mM |
| Menadione (vitamin K) | 0.2 | mg/l | 1.162 | μM |
| BSA (Bovine Serum | 7350 | mg/l | X | |
| Albumin) | | | | |
| CAA (Casamino Acid; | 5000 | mg/l | X | |
| hydrolysed casein with | | | | |
| low iron and NaCl | | | | |
| concentration) | | | | |
| Adenine | 1.35 | mg/l | 10 | μM |
| Flavin Adenine | 1 | mg/l | 1.21 | μM |
| Dinucleotide | | | | |
| Folinic acid | 1 | mg/l | 1.96 | μM |
| Pyridoxal phosphate | 5 | mg/l | 20.23 | μM |
| Dibasic fumarate | 500 | mg/l | 3.12 | mM |
| Pyruvate | 550 | mg/l | 5 | mM |
| Thiamine PyroPhosphate | 25 | mg/l | 54.26 | μM |
| Inosine | 2.7 | mg/l | 10.07 | μM |
| Mixture of volatile | 10 | μl/l | X | |
| fatty acids (valeric | | | | |
| acid, isobutyric acid, | | | | |
| isovaleric acid) | | | | |
| Coenzyme A | 1 | mg/l | 1.30 | μM |
| Protoporphyrin IX | 5 | mg/l | 9 | μM |
| FeSO4 | 1.66 | mg/l | 6 | μM |

The strain of S. gordonii was grown in liquid medium defined for its growth, the MMBC-4 medium, composed of all the elements of MMBC-S medium presented in Table 1 and the various additions presented in Table 2.

TABLE 2
Additions for the medium MMBC-4 defined for
the growth of Streptococcus gordonii

| Elements | Final concentrations | |
|---|---|---|
| D-Biotin | 0.05 | μM |
| Nicotinic Acid | 0.04 | mM |
| L-Glutamic Acid | 4 | mM |
| L-Arginine HCl | 1 | mM |
| L-Tryptophane | 0.1 | mM |
| MnSO₄ | 10 | mg/l |
| (NH₄)₂SO₄ | 0.6 | g/l |
| D-Glucose | 6 | g/l |

All the strains used in the study were cultivated under anaerobic conditions in a chamber thermostatically controlled to 37° C. (Modular Atmosphere Controlled System 5000, Don Whitley Scientific, Shipley, UK) containing a gaseous mixture of 10% hydrogen, 10% carbon dioxide and 80% nitrogen, or in airtight containers containing sachets generating anaerobic atmosphere (Anaerogen Oxoid).

Arenicola marina hemoglobin (HbAm) is supplied at a concentration of 24 g/L and diluted in a stabilization buffer, the composition of which is presented in Table 3.

TABLE 3
Composition of the HbAm stabilization buffer

| Elements | Final concentrations | |
|---|---|---|
| NaCl | 90 | mM |
| Na-gluconate | 23 | mM |
| Na-acetate | 27 | mM |
| KCl | 5 | mM |
| MgCl₂ | 1.5 | mM |
| CaCl₂ | 2.5 | mM |

2) Evaluation of the Effect of HbAm on Bacterial Survival

The experiment was carried out anaerobically in vials provided with a stopper allowing the injection of HbAm via a syringe through a sealed septum, this is in order to keep the anaerobic bacteria in the thermostatically-controlled enclosure, while keeping as much as possible the oxygenation of the HbAm inside the syringe prepared in aerobic condition. The solution containing the molecule was injected directly into the bacterial culture. MMBC-S or MMBC-4 media are inoculated at an initial OD600 nm of 0.1 in a final volume of 2 ml. The cultures were previously incubated anaerobically for 1 h30 or 24 h respectively for P. gingivalis or T. denticola, so that they are under optimal conditions for the continuation of the survival test. Subsequently, 2 g/L of HbAm (charged with oxygen) diluted in the stabilization buffer were injected through the septum of the vials; an equivalent volume of stabilization buffer (tsHbAm) or unreduced MMBC-S/MMBC-4 medium (kept in aerobic condition) was used for the controls. The experiments were carried out in triplicate. Several methods were used to quantify viable cells:

a) Enumeration on Columbia Blood Agar (S. gordonii and P. gingivalis)

After various culture times at 37° C. in anaerobic conditions (0 minutes, 30 minutes, 2 hours and 4 hours after injection of HbAm), the counting of the CFUs was carried out by depositing drops of diluted sample on Columbia blood agar. (2 serial dilutions per sample, three deposits per dilution), and counting of the colonies at the level of the deposits after 24 h of incubation at 37° C. anaerobically. Three independent experiments were carried out.

b) LIVE/DEAD Marking and Analysis by Fluorimetry (P. gingivalis and T. denticola)

In order to verify the effect of HbAm on the survival of bacteria, LIVE/DEAD labeling experiments making it possible to determine cell viability were carried out after 4 hours of anaerobic incubation of bacterial cultures, treated or not with HbAm. This test is based on the following characteristics: the fluorescent marker Syto 40 (5 μM) marks all of the cells, while propidium iodide (30 μM) marks only dead cells. This labeling was carried out in the samples after elimination of the culture medium by centrifugation at 7000 g for 10 minutes at 20° C., then washing of the bacteria in PBS. Controls of 100% living bacteria (without treatment injection) or 100% dead bacteria (by using a 70% ethanol treatment) were carried out in parallel with the samples in order to carry out calibration ranges for the percentage of live cells. The fluorescence of the two different markers was measured spectrofluorometrically at the excitation and emission wavelengths of 380 nm and 440 nm respectively, for the Syto 40 and at the excitation and emission wavelengths of 480 nm and 650 nm respectively for propidium iodide, using the POLARstar OMEGA plate reader (BMG LABTECH). The ratios of the fluorescence intensities of Syto 40 to those of propidium iodide were then calculated in order to analyze the bacterial survival in the presence of HbAm.

3) Study of the Expression of spxB in S. gordonii

a) Bacterial Culture

Cultures of S. gordonii were taken in the exponential growth phase and diluted to a third in an RNA stabilizing agent (RNAprotect Bacteria Reagent, Quiagen), incubated for 5 minutes at room temperature and then centrifuged for 10 minutes at 5000 g before freezing of the pellets at −80° C.

b) DNA Extraction

The bacterial pellets were resuspended in TE buffer (10 mM Tris-HCl, 1 mM EDTA, pH 8) containing 20 mg/ml of lysozyme and 2 mg/ml of proteinase K in order to carry out their enzymatic lysis. After an incubation of 30 minutes at 37° C., the mixtures were deposited on acid-treated glass beads (Sigma-Aldrich) and subjected to 3 cycles of 45 seconds at a speed of 6.5 m/s (Fastprep® FP120 Cell Disrupter, BIO 101 ThermoSavant), so as to perform a mechanical lysis of the bacterial cells. A 10 second centrifugation at 13000 g was then carried out and the supernatants were collected. The bacterial RNAs were extracted from the lysates obtained using the NucleoSpin® RNA kit (Macherey-Nagel) according to the supplier's recommendations. The DNA was removed from samples by digestion using DNase RQ1 (Promega) according to the supplier's recommendations. The RNA was then purified on a column by the RNA Clean & Concentrator™ kit (Zymo Research) according to the supplier's recommendations, and was assayed by spectrophotometry at 260 nm (Nanodrop MD-1000® ThermoScientific). In order to verify the absence of DNA in the purified extracts, a PCR (see below) was carried out using specific primers for the 16S DNA of each bacterial species (Table 4).

c) Amplification by Polymerase Chain Reaction (PCR)

The PCR reactions were carried out according to the protocol of the supplier of the One-Taq® polymerase 25 U/μL (NEB) with a C1000 thermocycler (BioRad). The reaction was carried out on a final volume of 25 μl, each tube containing 12.5 μl of the One-Taq 2× Mix, 0.5 μl of each of the primers R and L (Table 4) concentrated to 5 μM, as well as 0.5 μl of the sample to be analyzed, all supplemented to 25 μl with sterile Rnase-free water.

The PCR was carried out as follows:
Initial denaturation stage: 30 s at 94° C., then
30 cycles of three successive stages of denaturation of 30 s at 94° C., hybridization of 30 s at 55° C., and elongation of 12 s at 68° C., then
Final elongation of 5 minutes at 68° C.

The PCR products were then demonstrated on 2% (w/v) agarose gel (Eurobio) produced with TAE buffer 50× migration buffer (Biosolve) in the presence of a DNA intercalator, DNA Dye NonTox (AppliChem). Size markers, DNA ladder 2 log (New England Biolabs), from 1 kB to 50 bp, were used to verify the size of the amplified sequence. The revelation and photography of the gels were carried out after exposure to UV (365 nm) on a UVIDOC transilluminator (UVITEC Cambridge).

d) Reverse Transcription (RT)

The RNA was transformed into cDNA by reverse transcription with the ProtoScript™ II Reverse Transcriptase kit (New England Biolabs) according to the supplier's protocol. The reaction was carried out with an RNA matrix having a concentration greater than 40 ng, in a mixture of 20 μl containing 1 μl of dNTP concentrated at 10 mM, 2 μl of primers Random Primer Mix (New England Biolabs) concentrated at 60 μM, 2 μl of 1,4-Dithiothreitol 0.1 M concentrated, 4 μl of 5× concentrated buffer, 1 μl of RNase inhibitor (murine RNAse inhibitor 40 U/μl), 1 μl of reverse transcriptase (200 U/μl), all supplemented to 20 μl with sterile RNA-free water (Ambion). The reaction mixture was incubated in a C1000 thermocycler (BioRad) according to a program consisting of an initial step of 5 minutes at 25° C., then 1 hour at 42° C., then inactivation of the enzymes at 80° C. for 5 minutes.

e) Quantification of DNA by qPCR

The list of primers used during this study is presented in Table 4. The qPCRs were performed with the SYBR GREEN/ROX kit (Eurogentec), according to the supplier's recommendations in a final volume of 12.5 μl, each tube containing 6.25 μl of SYBR GREEN 2× Mix, 1 μl of each of the primers R and L concentrated at 5 μM, as well as 1 μl of the sample to be analyzed between 0.001 ng/μl and 10 ng/μl, all supplemented at 6.25 μl with sterile RNase-free water. The amplification reactions were carried out on a StepOne Plus device (Applied Biosystems), according to the following program: 2 minutes at 50° C. 10 minutes at 95° C., 40 cycles composed of a denaturation step of 15 s at 95° C., a hybridization and elongation step of 60 s at 60° C. A melting curve was produced after each amplification (15 seconds at 95° C., one minute at 60° C. followed by a temperature gradient from 60 to 95° C. and 15 seconds at 95° C.). The efficiencies of the primers RTSgo and spxB were determined by qPCR on a calibration range of SS 16S DNA using the formula 10^{-1/slope}−1. The differences in expression of the gene studied, spxB (primers spxB, Zheng et al., 2011) were calculated relatively by the 2-mct method by normalizing with the expression of the housekeeping gene of S. gordonii, rpoB, gene encoding the beta subunit of RNA polymerase (RTSgo primers, Park S. N. & Kook, J. K., 2013).

Table 4
Primers used

| Names | Sequence 5′ => 3′ |
|---|---|
| RTSgo F | TGTACCCCGTATCGTTCCTG |
| | TG (SEQ ID NO: 1) |
| RTSgo R | AAAGACTGGAGTTGCAATGT |
| | GAATA (SEQ ID NO: 2) |
| spx6 F | GGATGCTTTGGCTGAAGAC |
| | (SEQ ID NO: 3) |
| spx6 R | GGACCACCTGAACCTACTG |
| | (SEQ ID NO: 4) |
| DNA 16S Sg L | AGCGTTGTCCGGATTTATTG |
| | (SEQ ID NO: 5) |
| DNA 16S Sg R | CATTTCACCGCTACACATGG |
| | (SEQ ID NO: 6) |
| DNA 16S Pg L | TGGGTTTAAAGGGTGCGTAG |
| | (SEQ ID NO: 7) |
| DNA 16S Pg R | CAATCGGAGTTCCTCGTGAT |
| | (SEQ ID NO: 8) |
| DNA 16S Td L | GGGCTACACACGTGCTACAA |
| | (SEQ ID NO: 9) |
| DNA 16S Td R | CGTGCTGATGTGCGATTACT |
| | (SEQ ID NO: 10) |

4) Evaluation of the Effect of HbAm on the Inhibition of the Growth of P. gingivalis by S. gordonii

The evaluation of the inhibition of the growth of P. gingivalis by S. gordonii, was carried out on solid medium from an adaptation of the method of Herrero et al., (2016). The BHI-rich medium was replaced by the MMBC-4 medium, a controlled medium defined for the growth of S. gordonii, so that the elements present in the rich medium do not interfere with the experiment.

This test is carried out by inoculating 7 μl of concentrated S. gordonii at 109 CFU/ml on agar medium, and incubating it anaerobically for 24 h, followed by an inoculation of 7 μl of concentrated P. gingivalis at 109 CFU/ml at 5 mm from the edge of the S. gordonii deposit. The agars were incubated anaerobically for 3 days before observation. The inhibition is evaluated by measuring the distance between the edge of the deposit formed by the inhibiting bacteria (S. gordonii) and the edge of the deposit formed by the target bacteria (P. gingivalis).

This experiment was carried out in two different ways. For the first experiment, S. gordonii was inoculated on 3 types of agar medium, MMBC-4 medium alone, MMBC-4 medium in the presence of 2 gl of HbAm, and MMBC-4 medium in the presence of an equivalent amount of tsHbAm stabilization buffer. For the second experiment, S. gordonii was incubated in liquid medium MMBC-4 alone, MMBC-4 containing 2 g/l of HbAm or MMBC-4 containing an equivalent amount of tsHbAm before inoculation on agar medium MMBC-4 as described previously.

5) Evaluation of the Effect of HbAm on Biofilms of S. gordonii, P. Gingivalis and T. denticola

a) Formation of Static Biofilms

The formation of static biofilms was carried out in culture chambers mounted on a pSlide coverslip, ibiTreat: #1.5 polymer coverslip (Ibidi). Sterile saliva (from several healthy volunteers), treated with 2.5 mM dithiothreitol, centrifuged for 5 minutes at 2500 rpm at room temperature, then filtered through a 0.22 μm membrane and diluted to ¼, was applied in the culture chambers for 30 minutes. After these 30 minutes, the surplus was removed in order to remove the elements of the saliva that were not adsorbed on the surface of the chamber.

A bacterial mixture composed of cultures of S. gordonii (OD600 nm=0.05), P. gingivalis (OD600 nm=0.1) and T. denticola (OD600 nm=0.1) in MMBC-4 medium, was then inoculated in the culture chambers and incubated for 24 h anaerobically at 37° C. A treatment of HbAm at 2 g/I for 1 hour or its equivalent in tsHbAm was then applied to the biofilms by injection of the solutions using a syringe in order to conserve the oxygenation of the molecule within the Anaerobic limits.

b) Marking and Observation

The culture supernatants from the biofilms were removed and the biofilms were washed with PBS. The labeling was then done using a solution composed of a mixture of Syto 40 (5 μM) and propidium iodide (40 μM). Biofilms were observed after 24 hours of growth on the “photonic microscopy” platform of the “Microscopy Rennes Imaging Center” (SFR BIOSIT, Rennes) using a Leica TCS-SP8 confocal microscope (Leica Microsystems, Wezlar, Germany). A 63× immersion lens was used for image capture and a zoom of 1.5 was applied. The fluorescence emitted by the Syto 40 marker of all the cells was detected by excitation at 405 nm, with collection of the fluorescence emitted between 420 and 475 nm. Excitation at 514 nm allows detection of labeling with propidium iodide of dead cells between 620 and 650 nm. The biofilm images were captured at 0.5 μm intervals and were scanned at a frequency of 400 Hz. Leica software (LAS AF V.2.2.1) was used to pilot the microscope and capture the images. The qualitative analysis of the images was carried out using the ImageJ V1.43m software, COMSTAT2 (Heydorn et al., 2000). The biomass was determined by the sum of the biomass evaluated, by labeling with Syto 40, and by labeling with propidium iodide.

c) Quantification by qPCR

The bacteria of the starting inoculum and the bacteria detached from the biofilm were centrifuged at 7000 g and 20° C. for 10 minutes with resuspension of the bacterial pellet in PBS in order to remove the MMBC-4 medium and the treatments. The sessile bacteria making up the biofilm were resuspended in PBS. The bacteria were then heated to 95° C. and then quantified by qPCR using species-specific 16S primers, as described above.

Results and Discussion

1) Evaluation of the Effect of HbAm on the Survival of S. gordonii, P. ginqivalis and T. denticola in Planktonic Cultures

a) Effect of HbAm on the Survival of S. gordonii

In order to evaluate the effect of HbAm on the growth and survival of S. gordonii, cultures of this bacterium were incubated for 4 h under anaerobic conditions, after injection or not of HbAm at time 0. At each time, the colonies formed on Columbia blood agar under different test conditions, were counted.

The results show that there is no significant difference in survival or growth of S. gordonii in the presence or absence of 2 g/l of HbAm from 0 minutes to 4 hours. In fact, the log curves of bacterial concentrations as a function of exposure time are equivalent between the culture conditions in MMBC-4 medium alone, in the presence of tsHbAm stabilization buffer, and in the presence of HbAm. The relative comparisons of the survival of S. gordonii in the presence of HbAm compared to exposure to tsHbAm confirm this.

Therefore, the growth and survival of S. gordonii in planktonic culture does not seem to be affected by HbAm or by tsHbAm.

b) Effect of HbAm on the Survival of P. gingivalis

In order to evaluate the effect of HbAm on the survival of P. gingivalis, cultures of this bacterium were exposed to the molecule for 4 h under anaerobic conditions. At each time, the colonies formed on Columbia blood agar and coming from the different test conditions, were counted.

The results show that the stabilization buffer for the HbAm molecule does not significantly affect the growth and survival of P. gingivalis. In fact, the counts expressed in log (CFU/ml) are similar with or without stabilization buffer, from 109 CFU/ml at the start of the experiment to 4.109 CFU/ml after 2 hours of exposure. There was no significant difference after 4 h of exposure with 6.3.109 CFU/ml in MMBC-S medium alone and 5.109 CFU/ml in the presence of tsHbAm. The stabilization buffer was therefore used as the only negative control in the three experiments subsequently carried out.

A decrease in the CFU concentration was observed after 2 hours of exposure for bacteria exposed to HbAm at 2 g/I (2.5.109 CFU/ml) compared to the control samples containing only the stabilization buffer tsHbAm (4.109 CFU/ml), i.e. 35% mortality with HbAm. The effect is amplified after 4 hours post-exposure with 49% mortality in the condition of exposure with HbAm. The HbAm molecule, therefore, has an effect on the survival or division of bacteria from 2 hours, and this effect is increased with the time of incubation.

In order to confirm the effect of HbAm on the viability of P. gingivalis, a complementary method was used, based on the use of fluorophores (“LIVE/DEAD” kit) capable (Syto 40) or not (propidium iodide) of penetrating through the polarized cytoplasmic membrane (living cells) to bind to DNA. The specific fluorescence linked to Syto 40 and to propidium iodide of P. gingivalis cells, whether or not treated with the HbAm molecule, was measured by spectrofluorimetry after incubation in the presence of fluorophores.

The results show that the injection into anaerobic culture of unreduced MMBC-S medium or of stabilization buffer does not seem to significantly affect the survival of P. gingivalis (MMBC-S: 96% of living cells; MMBC-S plus tsHbAm: 93.9% of living cells). However, HbAm seems to have an effect on the cell viability of P. gingivalis since 4 hours after the injection of the molecule the level of living cells is only 60%. These results confirm the data obtained by counting the colonies.

HbAm Therefore has a Bactericidal Effect on P. gingivalis.

c) Effect of HbAm on the Survival of T. denticola

Obtaining colonies of T. denticola is tedious, non-reproducible and requires the use of complex media. As a result, the counting method is not suitable for assessing the survival of T. denticola in the presence of HbAm.

The colony counting method and the fluorimetric method with LIVE/DEAD probes allow concordant results to be obtained for P. gingivalis. The LIVE/DEAD method was therefore used for T. denticola.

Neither the stabilization buffer (92% of living cells) nor the unreduced MMBC-S medium (94% of living cells) appears to affect the viability of T. denticola. In contrast, the HbAm molecule significantly decreases the ratio of living cells to 70% in cultures 4 hours after injection of the molecule.

HbAm Therefore has a Bactericidal Effect on T. denticola.

2) Evaluation of the Effect of HbAm on the Production of H₂O₂ by S. gordonii

a) Effect of HbAm on the Inhibition of the Growth of P. gingivalis by S. gordonii

S. gordonii is known to produce H₂O₂ by pyruvate oxidase SpxB in the presence of oxygen and pyuvate. Furthermore, H22 is toxic to P. gingivalis. In order to determine whether the oxygen supply by HbAm allows an increase in the inhibitory properties of S. gordonii, inhibition experiments on agar medium were carried out. The results show that in agar media MMBC-4 alone, or containing 2 g/l of HbAm, or with an equivalent volume of tsHbAm stabilization buffer, the inocula of P. gingivalis incubated near S. gordonii seem to have a cell density that is weaker than P. gingivalis inocula incubated alone for the same media. In addition, the addition of 2 g/l of HbAm in the agars amplifies the inhibitory effect of S. gordonii on P. gingivalis since no colony of P. gingivalis is observable in this condition. The same remarks may be made for the experiment carried out on an agar medium MMBC-4 not supplemented, but with or without addition of HbAm or tsHbAm in the culture of S. gordonii before depositing on the agar.

S. gordonii therefore seems to produce greater inhibitors of P. gingivalis growth when exposed to concentrated HbAm at 2 g/l.

b) Effect of HbAm on the Expression of the spxB Gene in S. gordonii

The gene encoding pyruvate oxidase is spxB. In order to check whether the oxygen supply by HbAm is sufficient to allow an increase in the expression of the gene, a quantification of the relative expression of this gene compared to the housekeeping gene rpoB was carried out by means of an RT-qPCR.

The effectiveness of the primers used for rpoB was low (89%).

However, the difference in expression of spxB with or without HbAm was greater than 10 times and is, therefore, probably significant.

According to the first results, the value of 2^{-ΔΔCt} is 14.2 on average, signifying an expression of spxB 14 times greater with HbAm. HbAm would, therefore, provide a sufficient quantity of oxygen to allow a significant increase in the expression of spxB in S. gordonii cultivated under anaerobic conditions.

These results, coupled with those presented on the production of P. gingivalis growth inhibitors, suggest that HbAm could increase the production of H₂O₂ in a culture previously incubated under anaerobic conditions.

3) Evaluation of the Effect of HbAm on a Mixed Biofilm Composed of S. gordonii, P. gingivalis and T. denticola

In order to evaluate the inhibitory potential of HbAm on an already established mixed biofilm, comprising S. gordonii, P. gingivalis and T. denticola, 24 h biofilms were incubated for 1 h in MMBC-4 medium alone, or in the presence of stabilization buffer or HbAm, before being analyzed:
by confocal microscopy allowing study of the film thickness, the biomass and the proportion in dead cells,
by qPCR to quantify the bacterial species in the biofilms. In order to assess the effect of HbAm on the detachment of cells from the biofilm, bacterial quantification by qPCR was also carried out on the supernatants obtained after a 1 hour exposure time to HbAm or to the tsHbAm stabilization buffer, or else after treatment with 70% ethanol.

According to the first results, the LIVE/DEAD overlays established by confocal microscopy show a stronger red coloration of the bacteria in MMBC-4 medium exposed to HbAm (1% live/99% dead) than for bacteria exposed only to the buffer. stabilization tsHbAm (8% alive/92% dead), which would suggest that the biofilm cells exposed to HbAm would have suffered damage to their membrane allowing propidium iodide to enter their cytoplasm. However, it should be noted that the percentage of dead cells without HbAm is high.

Under our experimental conditions, therefore, the LIVE/DEAD test does not allow a definitive conclusion to be drawn as to the cell mortality of the bacteria in the biofilm specifically due to HbAm.

According to the analysis of images obtained by confocal microscopy, the biomass as well as the average thickness of the biofilm is lower after treatment of the biofilm with HbAm, than for treatments with the solubilization buffer alone (tsHbAm) or with 70% ethanol.

In addition, the proportion of cells detached from the biofilm after treatment is higher after treatment with HbAm (44%) compared to tsHbAm alone (2%). Treatment with 70% ethanol also induces significant detachment of bacteria with 51% of cells detached in this condition. In addition, bacterial quantifications show that with tsHbAm and especially HbAm, the species found in cells detached from the biofilm are mainly T. denticola and P. gingivalis whereas for biofilms treated with 70% ethanol, it is S. gordonii which is found in high percentage in the detached cells. HbAm would therefore cause the detachment of cells from the biofilm, and would have an effect mainly on P. gingivalis and T. denticola. These results are to be confirmed.

Regarding the bacterial composition of the different biofilms under the conditions studied, a high proportion of S. gordonii is found compared to P. gingivalis and T. denticola, whether in the presence of tsHbAm stabilization buffer (98.5%), HbAm (99.8%) or 70% ethanol (81.7%). The bacterial species of the starting inoculum were quantified by qPCR: 9.26.107 CFU/ml of S. gordonii (42.9%), 5.11.107 CFU/ml of P. gingivalis (23.2%), and 7.34.107 CFU/ml of T. denticola (33.9%). S. gordonii is in the majority in biofilm despite an innoculum made up of more than 60% of P. gingivalis and T. denticola.

In addition, it may be noted that in the presence of HbAm, a lower concentration of T. denticola and P. gingivalis is found within the biofilm compared to the control exposed to tsHbAm and to ethanol 70%.

In fact, a reduction of 10 CFU/ml in concentration of P. gingivalis and T. denticola was observed for the biofilms exposed to HbAm compared to the tsHbAm control. Therefore, there would be a detachment of these bacteria after exposure to HbAm.

1. Method for using a molecule chosen from among oxygen transporters of marine invertebrate animals as a bactericidal agent against Gram-negative bacteria that are pathogenic for humans.

2. Method according to claim 1, characterized in that it is chosen from among a globin of Annelids, a globin protomer of Annelids, or an extracellular hemoglobin of Annelids.

3. Method according to claim 1, characterized in that the Gram negative bacteria which are pathogenic for humans, are strictly anaerobic bacteria, more preferably this is P. gingivalis and/or T. denticola.

4. Method according to claim 1, for preventing and/or treating periodontal disease in a subject, comprising administering to said subject at least one molecule chosen from among oxygen transporters of marine invertebrate animals.

5. Method according to claim 4, characterized in that the periodontal disease is chosen from among gingivitis, periodontitis, periodontal recessions and periodontal abscesses.

6. Method for preventing and/or treating halitosis in a subject, comprising administering to said subject at least one molecule chosen from among oxygen transporters of marine invertebrate animals.

7. Method according to claim 6, characterized in that it is chosen from a globin of Annelids, a globin protomer of Annelids, or an extracellular hemoglobin of Annelids.

8. Method for whitening teeth of a subject, comprising administering to said subject at least one molecule chosen from among oxygen transporters of marine invertebrate animals, for use for whitening teeth.

9. Method according to claim 8, characterized in that it is chosen from among a globin of Annelids, a globin protomer of Annelids, or an extracellular hemoglobin of Annelids.

10. Method according to claim 2, characterized in that the extracellular hemoglobin of Annelids is chosen from among the extracellular hemoglobins of Annelid Polychetes.

11. Method according to claim 2, characterized in that the extracellular hemoglobin of Annelids is chosen from among the extracellular hemoglobins of the Lumbricidae family, the extracellular hemoglobins of the Arenicolidae family, and the extracellular hemoglobins of the Nereididae family.

12. Method according to claim 2, characterized in that the extracellular hemoglobin of Annelids is chosen from among the extracellular hemoglobin of Lumbricus terrestris, the extracellular hemoglobin of Arenicola sp, and the extracellular hemoglobin of Nereis sp.

13. Method according to claim 2, characterized in that the extracellular hemoglobin of Annelids is chosen from among the extracellular hemoglobin of Arenicola marina, and the extracellular hemoglobin of Nereis virens.

14. Method according to claim 2, characterized in that the globin, the globin protomer, or hemoglobin is present in a composition comprising a buffer solution and devoid of hydrocolloid, preferably in an aqueous solution comprising salts, and giving the composition a pH of between 6.5 and 7.6, or in a composition in the form of a gel.


SEQUENCE LISTING (10 sequences)

SEQ ID NO: 1 - DNA, 22 bp, Artificial Sequence
  primer RTSgo F
tgtaccccgtatcgttcctgtg

SEQ ID NO: 2 - DNA, 25 bp, Artificial Sequence
  primer RTSgo R
aaagactggagttgcaatgtgaata

SEQ ID NO: 3 - DNA, 19 bp, Artificial Sequence
  spxB F
ggatgctttggctgaagac

SEQ ID NO: 4 - DNA, 19 bp, Artificial Sequence
  spxB R
ggaccacctgaacctactg

SEQ ID NO: 5 - DNA, 20 bp, Artificial Sequence
  primer ADN 16S Sg L
agcgttgtccggatttattg

SEQ ID NO: 6 - DNA, 20 bp, Artificial Sequence
  primer ADN 16S Sg R
catttcaccgctacacatgg

SEQ ID NO: 7 - DNA, 20 bp, Artificial Sequence
  primer ADN 16S Pg L
tgggtttaaagggtgcgtag

SEQ ID NO: 8 - DNA, 20 bp, Artificial Sequence
  primer ADN 16S Pg R
caatcggagttcctcgtgat

SEQ ID NO: 9 - DNA, 20 bp, Artificial Sequence
  primer ADN 16S Td L
gggctacacacgtgctacaa

SEQ ID NO: 10 - DNA, 20 bp, Artificial Sequence
  primer ADN 16S Td R
cgtgctgatgtgcgattact